(19) United States
(12) Patent Application Publication
(10) Pub. No.: US 2019/0134140 A1
(43) Pub. Date: May 9, 2019

(54) ORALLY ADMINISTERED COMPOSITION FOR TREATING CYSTIC FIBROSIS, COPD, ASTHMA AND OTHER INFLAMMATORY CONDITIONS

(71) Applicant: Lauranell Harrison BURCH, Durham, NC (US)

(72) Inventor: Lauranell Harrison BURCH, Durham, NC (US)

(21) Appl. No.: 15/803,608

(22) Filed: Nov. 3, 2017

(51) Int. Cl. A61K 36/9066; A61K 9/00; A61K 31/12; A61K 31/05; A61K 36/70; A61K 31/352; A61K 36/23; A61K 36/489; A61K 31/4525; A61K 36/67; A61K 36/28; A61K 31/357; A61K 36/82; A61K 31/353; A61K 36/734; A61K 31/385; A61K 36/14; A61K 36/53; A61K 31/19; A61K 36/324; A61K 31/704; A61K 36/77

(52) U.S. Cl. CPC A61K 36/9066; A61K 36/77; A61K 31/12; A61K 31/05; A61K 36/70; A61K 31/352; A61K 36/23; A61K 36/489; A61K 31/4525; A61K 36/67; A61K 36/28; A61K 31/357; A61K 36/82; A61K 31/353; A61K 36/734; A61K 31/385; A61K 36/14; A61K 36/53; A61K 31/19; A61K 36/324; A61K 31/704; A61K 9/0053

(57) ABSTRACT

The invention relates to a new orally administered composition, which in present embodiments is a standardized phytochemical formulation but other embodiments may contain designed molecules. This formulation is capable of suppressing inflammation in a variety of disease conditions having an underlying inflammatory component, including but not limited to cystic fibrosis, COPD, asthma, and pancreatitis. It is an objective of the invention to provide a non-toxic therapy, which exhibits anti-oxidant anti-inflammatory properties without deleterious side effects.

### TECHNICAL FIELD

The present invention is a method of treating and/or managing cystic fibrosis, COPD, asthma and other inflammatory diseases in a subject in need of such treatment, by orally administering a formulation derived from this invention. The treatment is intended to reduce inflammation in the body that contributes to the disease processes in need of treatment, and/or to modulate the function of the cystic fibrosis gene, CFTR, an ABC transporter, in a manner which increases the beneficial roles of CFTR in reducing inflammation and excessive and sticky mucus production at mucosal surfaces throughout the body and/or in preventing infection of the respiratory tract mucosal surfaces including the sinuses. The invention is disclosed below, along with compounds and compositions useful for carrying out such methods.

### BACKGROUND

Cystic fibrosis (CF) is the most common fatal genetic disease among Caucasians. Although the clinical features of cystic fibrosis involve multiple organs, the primary cause of morbidity and mortality is chronic pulmonary infections. Cystic fibrosis (CF) is caused by mutations in a single gene: the cystic fibrosis transmembrane regulator (CFTR). CFTR controls transport of multiple ions responsible for proper hydration and the anti-inflammatory and/or antimicrobial defense of mucosal surfaces throughout the body. Loss of CFTR function results in accumulation of viscous secretions and repeated lung infections. Simply stated, cystic fibrosis airways are highly susceptible to microbial infection and inflammation while non-CF airways are resistant. As CF is a disease resulting from mutations in a single gene, the dramatic CF vs non-CF represent loss of functions directly attributable to and controlled by CFTR.

Currently there is no effective drug to prevent disease progression for most CF mutations. Standard CF therapy relies heavily on repeated use of antibiotics that ultimately fail to eradicate lung infection and lead to emergence of multi-drug resistant pathogens. Decline in lung function among CF patients is seen even in early childhood, and leads to requirement for lung transplant or premature death by respiratory failure.

Among substrates known to be dependent, entirely or in part, on CFTR for transport to mucosal surfaces are glutathione, bicarbonate and thiocyanate, all of which serve critical roles in airways defense against inflammation and infection. These findings of transport dependence of multiple large multi-atomic ions by CFTR should not be surprising, as cloning and sequencing of CFTR in 1989 revealed that CFTR is a member of the ABC transporter gene family.

Genes of the ABC transporter family actively transport multi-atomic molecules across a membrane in one direction using the energy of ATP to do the ‘pumping’. Originally, CFTR was believed to function mainly as a chloride channel. However, there are some CFTR mutations, called chloride-conducting mutants, which move chloride ions normally but still cause CF disease. If patients can move chloride ions through CFTR normally and still have severe and progressive CF lung disease, this must mean that transport of the multi-atomic substrates by CFTR (such as bicarbonate, glutathione, and thiocyanate), in other words the ABC transporter functions of CFTR, are essential to defense against inflammation and infection.

As healthy people with normal CFTR function are resistant to infection of the conducting airways, and patients with mutant CFTR are extremely susceptible to such infections, reducing inflammation in the body and restoring CFTR function through by use of natural or artificial CFTR modulating compounds represent key therapeutic goals for CF lung disease in particular, and for other disease where enhanced CFTR function may serve to reduce inflammation, which damages cells and tissues. Injured cells release mediators of inflammation which serve to perpetuate the inflammatory cycle. Therefore treatment with compounds that successfully reduce inflammation, even apart from CFTR modulation, is useful to break the inflammatory cycle.

Reduction in inflammation is associated with improved lung function even in normal individuals. Patients on statins, which are known to be anti-inflammatory, show less age related lung function decline than age matched controls not taking statins.¹ Subsequently it was shown that use of statins among lung transplant patients is associated with better graft survival and function.²

In addition to providing anti-inflammatory benefits, compositions resulting from this invention include ingredients demonstrated to be CFTR modulators. CFTR may be a useful therapeutic target for mucosal surface diseases other than CF, such as COPD, chronic bronchitis, pancreatitis, asthma and irritable bowel syndrome (IBS). There is growing evidence that other inflammatory lung diseases such as COPD and chronic bronchitis may be caused by acquired CFTR deficiency through damage of the epithelial surface where CFTR resides; this absence of functional CFTR contributes to the disease process.^{3,4} For this reason, compositions based on this invention may improve health outcomes for lung disease with an inflammatory component where other strictly anti-inflammatory treatment approaches have failed.

Defective or insufficient CFTR may be ‘corrected’ (restored to the cell surface) or ‘potentiated’ (increased in activity) by a variety of natural molecules derived from plants. Alternatively, synthetic molecules may be used. Plant-derived polyphenolic compounds are the preferred CFTR modulating molecules due to lower risk of toxicity. Molecules may be provided by oral administration in an amount and combination effective to cause correction and/or potentiation of CFTR at the tissue site in need of treatment.

Combinations of several correctors and/or potentiators are known in the art to be more efficacious than any single molecule to modulate CFTR function. Some single molecules may serve both to correct and potentiate CFTR. Ideally, molecules that have both anti-inflammatory and CFTR modulating ability are used in this invention. Natural compounds, historically, tend to be safer than synthetic drugs, particularly when food-derived molecules with a long history of safe dietary use are selected. Reducing toxicity of anti-inflammatory and CFTR modulating therapies is one of the specific problems of the prior art that this invention addresses.

### SUMMARY

The invention described here, when used in the proper composition and dose to reduce inflammation and/or to modulate CFTR function, is useful to treat cystic fibrosis, other airway diseases with an inflammatory component, and other diseases of mucosal surfaces with an inflammatory component.

Airways diseases that may be treated by this invention may include but are not limited to: inflammatory lung disease, pulmonary vasculitis, pulmonary sarcoidosis, inflammation and/or infection associated with lung transplantation, acute lung graft rejection, bronchiolitis obliterans syndrome (BOS), pulmonary artery hypertension (PAH), bronchitis, chronic bronchitis, sinusitis, asthma, cystic fibrosis, airways bacterial infection fungal infection, airways parasite infection, airways viral infection, chronic obstructive pulmonary disease (COPD), persistent pulmonary hypertension of the newborn (PPHN), primary ciliary dyskinesia (PCD), alveolar proteinosis, idiopathic pulmonary fibrosis (IPF), familial pulmonary fibrosis (FPF), eosinophilic pneumonia and eosinophilic bronchitis, acute respiratory distress syndrome (ARDS), mechanical ventilation-associated inflammation and/or infection, ventilator-associated pneumonias, asbestos-related airway disease, dust-related airway disease, silicosis, chemical agent-related airway disease and any combination thereof.

Compositions from this invention that are currently in use as dietary supplements have resulted in rapid double digit increases in FEV1 among users with cystic fibrosis and also reduction of GI symptoms characteristic of cystic fibrosis as well as other symptoms characteristic of cystic fibrosis in these same patients.

The invention may be used to treat other mucosal surface disease with an inflammatory component, such as but not limited to: irritable bowel syndrome (IBS), and pancreatitis. The invention may also be used to treat any and all other diseases where inflammation is an underlying factor in pathology.

This composition of this invention reflects the fact that mechanisms controlling inflammation and CFTR modulation are complex and interdependent and not likely to be addressed well by a single molecule. Therefore, this invention is a combination formulation comprised of anti-inflammatory/antioxidant molecules that can be demonstrated to reduce inflammation and/or to directly or indirectly modulate CFTR function. Ideally molecules used in this invention are both anti-inflammatory and CFTR modulating.

Among inflammatory lung diseases, the invention described here can be used to delay or even prevent deterioration of lung function, development of bronchiectasis, cough, dyspnea, excessive mucus production, chronic airways infection, and ultimately respiratory failure. No currently available natural or pharmaceutical CFTR modulator therapy demonstrates ability to prevent decline in FEV1 in patients with cystic fibrosis. There is also no current effective disease modifying therapy to prevent lung function decline in pulmonary fibrosis and COPD.

The present invention provides a method of treating and/or managing inflammatory and/or infectious diseases by administering a combination drug therapy. By “treating” or “managing” it is meant improving, preventing worsening of, and/or alleviating symptoms of a disease.

In particular, treating cystic fibrosis includes causing one or more of the following: increasing FEV1, increasing blood oxygen saturation, enhanced CFTR activity, augmented airway hydration, improving digestion, increasing pancreatic function, raising airway surface liquid pH, reducing inflammation in the airways, improved mucociliary clearance, bronchodilation, and antimicrobial effects. The present invention may be used to defend against inflammation and infection at mucosal surfaces other than airway surfaces. Such other mucosal surfaces include gastrointestinal surfaces, oral surfaces, sinuses and nasal surfaces, genitourinary surfaces, ocular surfaces or surfaces of the eye, the inner ear, and the middle ear.

The present invention may be used to defend against inflammation at tissues other than mucosal surfaces. Such tissues may include but are not limited to vascular tissues.

The present invention is primarily concerned with treatment of human subjects, but may also be employed for treatment of other mammals.

The first aspect of the invention is use of compound(s) that reduce inflammation in the body. Compounds may have direct antioxidant effects and/or work indirectly to reduce production of pro-inflammatory cytokines and/or other mediators of inflammation. Certain plant phytochemicals are well known to reduce inflammation in the body and may serve to reduce inflammation when incorporated in this invention. Below are a few examples but not a complete list of compounds that may be used in this invention. Green tea extract contains multiple beneficial molecules such as epigallocatechin gallate (EGCG). Green tea extract is an herbal derivative from the leaves of Camellia sinensis.

Escin is a pentacyclic triterpene isolated from the flowering tree Aesculus hippocastanum, commonly known as horse chestnut. Used safely for many years to treat inflammatory and vascular conditions, Escin is available as a therapy in many countries under a variety of brand names.

The pentacyclic triterpenes boswellic acids are found in the resin of the plant Boswellia serrata. Like escin, Boswellic acids are widely used herbal therapies. Also known as frankincense, Boswellia is an especially ancient therapy used to treat pain and inflammation. Interestingly, Ali and Mansour reported in 2011 that oral administration of Boswellic acids attenuates pulmonary fibrosis in a mouse model of this disease; notably for CF, in part through suppression of neutrophil migration, suppression of the pro-inflammatory mediator TGF-B, and through inhibition of 5-Lipoxygenase (5-LOX), preventing lung injury.⁵

Resveratrol is a promising CFTR corrector/potentiator^{6,7} already used by patients, with good responses reported anecdotally by patients homozygous for the most common CF mutation, dF508. However, resveratrol is poorly absorbed through the gut. Resveratrol is reported to receive enhancement in permeability when combined with quercetin (310%), curcumin (300%), and piperine (350%).⁸

Naringin is a flavone glycoside found naturally in citrus fruits. Naringin is structurally similar to apigenin, one of the most potent CFTR activating natural compounds used by researchers in drug screening experiments.^{9,10} Naringen demonstrates protective effects in diabetes and elevated oxidative stress,^{11, 12} and is believed to act, like apigenin, by increasing CFTR activity.¹³ Indrepta A now substitutes apigenin for naringen, to alleviate concerns of some patients regarding citrus ingredients.

Quercetin is demonstrated to activate dF508¹⁴ and is also reported to increase ciliary beat frequency, suggesting its potential to help patients with chronic rhinosinusitis.¹⁵ Silymarin from milk thistle has been administered for more than 2,000 years, primarily to treat liver dysfunction.¹⁶ The German Commission E for natural compounds recommends milk thistle use for dyspeptic complaints, toxin-induced liver damage and as supportive therapy for chronic inflammatory liver conditions.¹⁷

Alpha Lipoic Acid (ALA) is a potent antioxidant. As a supplement, it has shown benefit against oxidative stress and inflammation. In vivo and in vitro studies demonstrate that ALA exhibits ability to scavenge free radicals, regulate the detoxification of heavy metals, and modulate various pathways in pathological conditions.¹⁹

Pycnogenol is derived from bark of the maritime pine tree (Pinus maritima) and its medicinal uses date back at least 2000 years. Pycnogenol is considered beneficial for wound healing and for reducing vascular inflammation. Pinus maritima bark extract contains active polyphenols including catechins, taxifolin, procyanidins, and phenolic acids. Pycnogenol inhibits TNFα-induced NF-KB activation, in addition to adhesion molecule expression in the endothelium; which may serve to limit neutrophil migration into airways in cystic fibrosis. Pycnogenol also statistically significantly inhibited the expression of inflammatory marker matrix metalloproteinase 9 (MMP-9). MMP-9 is highly expressed at sites of inflammation and contributes to pathogenesis of various chronic lung diseases.

Amentoflavone is a biflavonoid of apigenin, one of the most potent natural CFTR potentiators. Amentoflavone is also known as bis-apigenin. Amentoflavone is demonstrated to be a phosphodiesterase inhibitor (PDE) that can break down enzymes that destroy cyclic AMP (cAMP), a molecular activator of CFTR.

Forskolin is an herbal compound which directly activates the adenylate cyclase enzyme, thereby generating cAMP from ATP and raising intracellular cAMP levels. Cyclic AMP (cAMP), is a molecular activator of CFTR. Forskolin is a mainstay CFTR-activating drug used by laboratory researchers as well as an ancient remedy used to support lung, heart, and urinary health, among numerous other uses. Forskolin increases cAMP, and then the other side of the coin, amentoflavone, is added to Indrepta B to prevent cAMP breakdown. The desired net effect of the combination of Amentoflavone and Forskolin is increased and extended overall CFTR activity.

Other natural plant sources with extract compounds that may be used to address inflammation in this invention include but are not limited to: Guggul, Holy basil, Crataegus pinnatifida bunge (Hawthorne), Neem, Boswellia serrata, Silybum marianum, Matricaria recutita (German Chamomile), Withania somnifera (ashwagandha), Zingiber officinale (ginger), Piper nigrum (black pepper), Sophora japonica, and Curcuma longa [curcumin]. Alternatively, ibuprofen may be used.

Astaxanthin is a carotenoid that gives carrots, salmon, shrimp and lobsters their orange color. Astaxanthin is a powerful antioxidant and may be used to reduce inflammation in this invention. Alternatively, n-acetyl cysteine or n-acetyl lysine may be used. Other aspects of this invention may contribute by various direct or indirect means to overall reduction of inflammation in a subject.

Another aspect of the invention comprises administering molecules that modulate CFTR function by correction and/or potentiation to enhance CFTR activity in the cell membranes where CFTR is normally expressed. Defective or insufficient CFTR may be corrected (restored to the cell surface) or potentiated (increased in activity) by a variety of phytochemical molecules that are natural plant extracts. Alternatively, CFTR may be modulated in other ways, for example by increasing cyclic AMP, a well-known activator of CFTR, within cells using compounds such as forskolin. Importantly, the beneficial activity of agents that increase cyclic AMP such as forskolin may be sustained in this invention by including compounds within the formulation which are phosphodiesterase inhibitors capable of inhibiting the breakdown of cyclic AMP, such as amentoflavone.

Synthetic molecules may also be used as anti-inflammatory agents and/or for CFTR modulation in this invention. However, plant-derived phytochemical compounds are the preferred anti-inflammatory and CFTR modulating molecules. Molecules are provided by oral administration in an amount and combination effective to reduce inflammation in the subject and/or to cause modulation of CFTR activity at target sites of inflammation in the body. Combinations of several correctors and/or potentiators are known in the art to be more efficacious than any single molecule to restore CFTR function. Molecules such as flavones and isoflavones, benzoflavones, flavonoids, xanthines, terpenes, pentacyclic triterpenes, stilbenes and benzimidazoles are among the preferred classes of molecules that may be used, but the invention is not limited to these classes. Curcuminoids, resveratrol, apigenin, EGCG, rutin, naringen, luteolin and quercetin are examples of molecules that may be used in this invention as modulators of CFTR function. Some molecules may serve both to correct and potentiate CFTR.

The present invention is explained in greater detail below.

### DETAILED DESCRIPTION

“(ABC)” means ATP Binding Cassette, as in ABC transporter.
“ABC transporter” means a molecule that uses the energy of ATP to transport substrates across a cell membrane.
“(ALI)” means acute lung injury.
“(ARDS)” means acute respiratory disorder syndrome.
“(CF)” means cystic fibrosis.
“(CFTR)” means the Cystic Fibrosis Transmembrane Regulator or CF gene
“(COPD)” means chronic obstructive pulmonary disease.
“(FEV₁)” means forced expiratory volume defined as the maximum amount of air expired in one second.
“(FPF)” means familial pulmonary fibrosis.
“(IPF)” means idiopathic pulmonary fibrosis.
“(PAH)” means pulmonary arterial hypertension.
“(PCD)” means primary ciliary dyskinesia.
“(PPHN)” means persistent pulmonary hypertension of the newborn.
“Airway surface” as used refers to airway surfaces below the larynx and in the lungs, as well as air passages in the head, including the sinuses, in the region above the larynx.
“Corrector or CFTR corrector” means a modulator of CFTR function that is a modulator of cellular processing and/or the localization the CFTR molecule within the cell.
“Disease” means any condition that damages or interferes with normal function of a cell, tissue, or organ.
“Inflammation” means a part of the complex biological response to harmful stimuli such as pathogens, cell damage, or irritants, and involves immune cells, blood vessels and molecular mediators.
“Potentiator or CFTR potentiator” means a modulator of CFTR function that increases CFTR function that is already properly localized within the cell.
“Subject” is defined to refer to any mammal requiring treatment. In the preferred embodiment, the subject is a human. The terms “human,” “patient,” and “subject” are used interchangeably hereafter.
“Modulate CFTR function” means to increase CFTR activity and/or stability and/or messenger RNA level.
“Mucosal surfaces” or “mucosa” are membranes that lines various cavities in the body and surround internal organs.
“Natural extract” as used herein denotes any extract that is obtained from a natural source, such as a plant, fruit, root, tree, and the like.
“Natural compound” as used herein denotes any individual molecule isolated from a plant or natural extract.
“Treat”, “treating”, and “treatment” refer to reducing a disease symptom and/or slowing progression of a disease.
“Reducing inflammation” means the ability to inhibit known biological pathways of inflammation under experimental conditions and/or to inhibit markers of inflammation and/or symptoms of inflammation in vivo, which include but are not limited to: pain, swelling, redness, and warmth, fatigue, headaches, muscle stiffness, inflammatory cell recruitment, tissue damage and vascular dysfunction.
“Active component” means formulation ingredient with a beneficial drug action and their salts or analogs.
“Effective amount” as used herein, means an amount of active ingredient sufficient to produce a selected effect.
“Anti-inflammatory activity” herein means activity as determined by any generally accepted in vitro or in vivo assay or test, for example an assay or test for any marker of inflammation, such as production of cytokines, prostaglandins or 8-isoprostane.
“Antioxidant activity” herein means activity as determined by any generally accepted in vitro or in vivo antioxidant assay or test.

In some embodiments, the combination drug based on this invention is administered via oral administration. Suitable dosages of the present invention can be determined depending on such factors as the nature and/or severity of the illness, frequency of administration, the duration of treatment, condition of the patient, the size and age of the patient, and any other relevant factors. One skilled in the art would also know how to monitor the treatment progress in order to determine an effective dose and treatment plan. For example, one skilled in the art could monitor patient spirometry, chest X-rays and CT's, sputum cultures and blood tests. The treatment may be administered as frequently as 3-5 times daily in order to obtain the desired therapeutic effect of treating the inflammatory disease.

Frequency of administration will depend, for example, upon the nature of the dosage form used and upon the severity of the condition being treated. The present invention may be administered in combination with other therapeutic agents that are administered orally or by other routes of administration. Such therapeutic agents can be administered before, after or concurrently with administration of the invention. Particular doses of any of these and other additional co-administered therapeutic agents can be determined by a physician or other qualified medical professional depending on factors such as the type of therapeutic agent, the nature and severity of the illness, route and frequency of administration, the duration of treatment, condition of the patient, size and age of the patient, and any other relevant factors.

Additional therapeutic agents can also be delivered by a vaporizer, humidifier or fogger. The foregoing descriptions have been supplied merely to illustrate the invention and are not intended to be limiting. Each disclosed aspect and embodiment of the present invention may be considered individually or in combination with any other aspects, embodiments and variations of the invention. Modifications of the disclosed embodiments incorporating the spirit and substance of the present invention may occur to persons skilled in the art and such modifications are within the scope of the present invention. Furthermore, all references cited herein are incorporated by reference in their entirety.

Ingredients described herein can serve a plurality of functions, thus disclosure of an ingredient herein as exemplifying one function or functional class does not exclude the possibility that it can also exemplify another function or functional class.

### Formulations and Administration

The active compounds disclosed herein may be orally administered to the subject by any suitable means. The oral composition is administered as capsules, chewables, tablets, powders, granules or liquid.

The formulations described herein may be co-administered with therapeutically active drugs, with other natural extract compounds and supplements, with vitamins or other compounds that may be advantageously utilized in a combination treatment according to the invention. The disclosed compositions maybe administered prior to administration of the known therapeutic, for example at least four hours prior to administration of the known therapeutic. Alternatively, the disclosed compositions may be administered concurrently with the known therapeutic provided there is no adverse interaction with the known therapeutic agent.

The following examples of the present invention in combination described in further detail, but the scope of the invention in any of these examples is not subject to restrictions.

Specific examples of active compounds that may be used to carry out the present invention are set forth below.

### Dosing Regimen

The dosage of the formulation will vary depending on the condition being treated and the state of the subject. Those of a skill in the art will appreciate that the preferred dosing regimen can be varied depending on symptoms, body weight, health and condition of the patient, and the like, and that the preferred dosing regimen can be readily determined using known techniques.

The dosing regimen requires either multiple daily administrations for a time limited to duration of the disease or conditions in need of treatment, such as in acute lung injury or acute pulmonary hypertension, or multiple daily and/or chronic administrations, particularly among chronic diseases, such as cystic fibrosis, COPD and primary ciliary dyskinesia.

### Efficacy Determination

The effect of the treatment may be clinically determined by measurements as described herein. Efficacy may be measured by the reduction of symptoms of inflammation and/or by other endpoints specific to the condition of the diseased subject. For lung diseases, efficacy of a therapy is measured by improvements in pulmonary function tests, improved oxygen saturation, improved quality of life and reduced frequency of exacerbations, however other endpoints may be desirable to include.

Patients with chronic lung diseases are closely monitored by regular clinic visits. Forced expiratory volume per one second (FEV1) will be measured regularly via spirometry, and also exacerbation rate, and blood O2 saturation. CF quality of life measures (CFQ-R), and exercise capacity are also useful clinical endpoints for lung diseases.

Specifically, treating and/or managing cystic fibrosis can include any one or more of the following: improved lung function, improved quality of life, reduced pulmonary exacerbation, reduced the microbial load, reversion of antibiotic susceptibilities of colonizing pathogens, improvement of the gastrointestinal tract and pancreatic function, and treatment of other mucus membranes of the body such as the sinuses.

Lung function may be improved by increasing the patient's forced expiratory volume in one second (FEV1), the forced vital capacity (FVC), and/or whole-lung mucus clearance. Lung function can be measured by spirometry or plethysmography. Lung function can also be assessed by measuring lung volume according to American Thoracic Society standards as described by the American Thoracic Society.

Pulmonary exacerbation is determined by clinical need for IV antibiotics and/or through presence of the following symptoms: change in sputum volume or color, new or increased hemoptysis, increased cough, increased dyspnea, malaise, fatigue or lethargy, a fever, anorexia or weight loss, sinus pain or tenderness, change in sinus discharge, change in findings on physical examination of the chest, decrease in pulmonary function from a previously recorded value, or radiographic change indicative of pulmonary infection.

Combination of the Formulation with Other Therapies

The formulations described herein provide a method to improve the pulmonary condition in cystic fibrosis and may, therefore, be effectively combined with other currently existing and known therapies. Individual therapeutic combinations, doses and specific formulations will depend on interaction with the other drug(s), which are expected to be minimal. The optimization of these combinations is based on the knowledge available in the art.

In particular, the formulation may be advantageously utilized in combination with a beta-agonist, steroid, anti-inflammatory agent, antibiotic, bronchodilator, mucolytic or another suitable drug. Safety endpoints for evaluation of this invention are: FEV1, systemic (blood) and urine levels of formulation active ingredients, liver enzymes, GI symptoms and other adverse events such as dyspnea, and chest tightness.

Efficacy endpoints for the determination of efficacy of the present invention are: pulmonary function (FEV1), and exhaled nitric oxide (NO). Chest X-rays and CT scans may also be taken. Exploratory endpoints for determination of efficacy include sputum expectoration culture.

### EXAMPLES

Products based on this invention were prepared in an FDA-licensed facility under GMP (Good Manufacturing Practices) and their formulations are supplied below. Because all ingredients used in these formulations are on the FDA GRAS list (Generally Recognized as Safe) for use in dietary supplements, the products were made available immediately for distribution to patients for voluntary personal use through funding by the Sharktank Research Foundation, a 501(c)3 nonprofit lung disease research organization. Products released to date, based on this invention, are known by the name Indrepta.

The A and B versions of Indrepta (see Supplement Facts tables) were released first. Most patients with some residual CFTR function were able to discern an added benefit of the forskolin/amentoflavone cyclic AMP component of the B version in their ability to breathe.

The following formulations resulting from the invention disclosed herein are effective to increase FEV1 by double digits in patients with cystic fibrosis, as evidenced by voluntarily shared clinical data from doctor visits. Patients also report reduction of inflammation as determined by reduced need for steroid use and ibuprofen, and reduction in other CF symptoms. Formulations based on this invention to date are safe and well-tolerated when orally administered in vivo.

Formulation A is composed solely of compounds which are anti-inflammatory and/or CFTR corrector/potentiators, and lacks a cyclic AMP activator strategy (eg. Forskolin) and augmentation by phosphodiesterase inhibitors (eg. Amentoflavone) that is utilized in the B and C formulations that are derived from this invention.

Supplement Facts
Serving size 3 capsules
Suggested use - 3 servings per day
Servings per container - 270

| | DELIVER |
|---|---|
| FORMULATION A | PER DAY (mg) |
| INGREDIENTS: | Per 9 capsules |
| Turmeric (Curcuma longa) 95% curcuminoids | 2000 |
| Trans-Resveratrol (Polygonum cuspidatum) 98% | 240 |
| Apigenin (Apium graveolens) 98% | 50 |
| Quercetin dihydrate (Sophora japonica) 95% | 200 |
| Bioperine ™ (Piper nigrum) 95% piperine | 10 |
| Milk Thistle Seed (Silybum marianum) 80% silymarin | 80 |
| EGCG (Camellia sinensis) 98% polyphenols, | 270 |
| 80% catechins, 50% EGCG | |
| Hawthorne fruit (Crataegus pinnatifida bunge) | 160 |
| 2% vitexins | |
| Hawthorne leaf (Crataegus pinnatifida bunge) | 160 |
| 2% vitexins | |
| Alpha Lipoic Acid (sodium) | 400 |
| Natural flavor blend | 900 |

### Example 1 Formulation A

Supplement Facts
Serving size 3 capsules
Suggested use - 3 servings per day
Servings per container - 270

| | DELIVER |
|---|---|
| FORMULATION B | PER DAY (mg) |
| INGREDIENT: | Per 9 capsules |
| Turmeric (Curcuma longa) 95% curcuminoids | 2000 |
| Trans-Resveratrol (Polygonum cuspidatum) 98% | 240 |
| Amentoflavone (Platycladus orientalis leaf) 20% | 150 |
| Quercetin dihydrate (Sophora japonica) 95% | 200 |
| Bioperine ™ (Piper nigrim) 95% piperine | 10 |
| Milk Thistle Seed (Silybum marianum) 80% silymarin | 80 |
| EGCG (Camellia sinensis) 98% polyphenols, | 270 |
| 80% catechins, 50% EGCG | |
| Hawthorne fruit (Crataegus pinnatifida bunge) | 160 |
| 2% vitexins | |
| Hawthorne leaf (Crataegus pinnatifida bunge) | 160 |
| 2% vitexins | |
| Forskolin (Coleus forskholii, 10% Forskolin) | 50 |
| Natural fruit flavor | 450 |

### Example 2 Formulation B

### Example 3 Formulation C

Formulation C is preferred with regard to reduction in symptoms by CF patients from most mutation classes who suffer from allergies, asthma or lung bleeds. These symptoms are reported by users to be improved significantly more by C vs B formulation. We do not observe that patients carrying dF508 mutations with a nonsense mutation show improved results with formulation C over patients carrying dF508 with a frameshift (a mutation that is unlikely to respond to CFTR modulation).

Supplement Facts
Serving size 3 capsules
Suggested use - 3 servings per day
Servings per container - 270

| | DELIVER |
|---|---|
| FORMULATION C | PER DAY (mg) |
| INGREDIENT: | Per 9 capsules |
| Turmeric (Curcuma longa) 95% curcuminoids | 2000 |
| Trans-Resveratrol (Polygonum cuspidatum) 98% | 240 |
| Amentoflavone (Platycladus orientalis leaf) 20% | 150 |
| Quercetin dihydrate (Sophora japonica) 95% | 200 |
| Bioperine ™ (Piper nigrim) 95% piperine | 10 |
| Milk Thistle Seed (Silybum marianum) 80% silymarin | 80 |
| EGCG (Camellia sinensis) 98% polyphenols, | 135 |
| 80% catechins, 50% EGCG | |
| Hawthorne fruit (Crataegus pinnatifida bunge) | 160 |
| 2% vitexins | |
| Hawthorne leaf (Crataegus pinnatifida bunge) | 160 |
| 2% vitexins | |
| Forskolin (Coleus forskholii, 10% Forskolin) | 50 |
| Boswellic acid (Boswellia serrata) 90% | 500 |
| Escin (Aesculus hippocastanum) 20% | 150 |

### Examples of FEV1 Improvements in Patients with Cystic Fibrosis Using Indrepta

### Patient 1:

21% FEV1 increase, 1 month on Indrepta
Adult female patient with cystic fibrosis, ddF508.
“Lung function last clinic fev1 was 1.14. Today they are 1.39 sooooo happy. Percentage before I started was 30. It's 51 now.”

### Patient 2:

15% FEV1 increase, approximately 1 month on Indrepta.
Female child with cystic fibrosis, age 6.
“Her last CF clinic 7/11: PFTs were 79%, Wt to ht percentile was 47%. Today at her CF clinic: PFTs are 94% and wt to ht percentile is 60%.”

### Patient 3:

14% FEV1 increase, 2 months on Indrepta
Adult female patient dF508 and 1209+1 G>A
“I'm hooked and so happy to report my lung function improvement”

### Patient 4:

7% FEV1 increase, approximately 2 weeks on Indrepta.
Adult male with cystic fibrosis, genotype unknown.
“Well, my vest has been broken and is being repaired. And I still blew 82%. Been getting up a ton of junk during treatments even without the vest (using the VibraLung, but nothing beats a good shakedown) Around 2 weeks (give or take) on Indrepta.”

### Patient 5:

21% increase in FEV1 after 7 weeks on Indrepta
Adult female dF508 and R758X
“Yes, I had a PFT jump from Indrepta. Yes, I'm clearing out a ton of mucus.
But can we get a hallelujah for waking up, not having a coughing fit, and not needing a breathing treatment?!”

### Patient 6:

12% increase in FEV1 after 3 months on Indrepta Adult female ddF508
“This was amazing news for us. In addition to that, probably 5 out of the last 6 years, she would get pulmonary infection flareup in the spring time around March or April, that would require antibiotic IVs. This year she hasn't had any issues with that.”

### Patient 7:

8.5% FEV1 increase after 5 weeks on Indrepta
Female child age 12 dF508 and 1717-1G>A
“Back at clinic with my daughter, and she gained 11b, 4 ozs in 2 weeks. That has never happened before! ETA: Her pft's went from FEV1—58% to 66.5%”

### Patient 8:

12% FEV1 increase in weeks, gained 16 pounds
Adult female dF508 1304delA
“Mine (PET'S) have gone up 12%! I feel so clear, my husband has realized I hardly ever cough, and my pleurisy is almost GONE! I had pleurisy for 6 months from a bad case of pneumonia and a chest tube placed. I became very discouraged but I totally feel like this drug is a God send. My mucus is so clear (my doctor couldn't believe it) and I am able to run miles at a time now. I got weighed again today and gained 4 more pounds. I am up 16 pounds since March. I couldn't believe the scale when I saw it. This is insane! I truly believe I am absorbing Fats now from Indrepta. I'm so excited. I am a few pounds away from my GOAL weight!”

### Patient 9:

15% FEV1 increase in 3 weeks
Adult female dF508 and g551s
“Pft's done. I'm up to 45%. Amazing! If you haven't tried Indrepta, you should! 6 weeks on indrepta c and it's went up 15%. Also I have a cold right now. Transplant talk has been pushed up due to improvement in lung function!!!!”
“I had been without indrepta for well over a week and started taking B yesterday thanks to a friend sending me a full bottle. I can tell a huge difference within the first couple of hours of the first three caps. I had been up all night with a terrible junk that was just stuck. My indrepta arrived around 1 pm. By 3 pm my mucus had started getting thinner and coming up. I'm a a critic of everything. I believe in science. I just can't say it enough, you need to try Indrepta.”

### Quality of Life Improvements

### Patient 5:

“I was diagnosed with folliculitis and dermatitis by the dermatologist earlier this year. My skin (back and shoulders) were completely broken out, and it was from bacteria and yeast. They tried antibiotics which didn't work. Bleach baths and SeneSerum-C helped a good bit. I've dealt with these issues for almost 2 years now although progressively worse at the end of 2016.
I took a back photo today and it is sooooo ridiculously clear since being on Indrepta”

### Patient 10:

“I'm proud to say that on August 14th I realized my nose was clear and no more yellow infection, my mucus is back to it's “normal” yellow and it's regular production!! My fevers have completely disappeared and I conquered a cold for the first time without IV antibiotics in over 10+ years!!! I'm completely amazed and thrilled!!”

### Patient 11:

Adult female age 42 dF508 and N1303K
“The first time I beat a respiratory infection was my first few weeks on indrepta. It was about 3 weeks last time but I was likely going through the normal mucus purge as well. In summary, in 3 months, I have beat 2 viruses, gained 2 pounds, have minimal mucus, am sleeping flat (not on pillow mtn) and have a lot more energy than I used to by end of day.
This makes me feel like I can be CF Ninja Warrior one day on American Ninja warrior . . . ”

The present invention is not to be limited in scope by specific embodiments described herein as examples, which are intended as single illustrations of individual aspects of the invention. Functionally equivalent methods and components not provided as examples are within the scope of the invention. Indeed, various modifications of the invention, in addition to those shown and described herein will become apparent to those skilled in the art from the foregoing description. Such modifications are intended to fall within the scope of the appended claims.

The invention is a composition suitable for treatment of a subject with an inflammatory condition in need of such treatment, wherein said composition is composed of combinations of entirely natural plant-derived molecules, or which may also include synthetic molecules in combination with natural plant-derived molecules, or be composed entirely of synthetic molecules, such that these molecules included in the formulation serve to reduce inflammation at the disease site in the subject and/or to modulate CFTR function in a manner specified in the claims.

Examples of suitable classes of molecules for this invention include, but are not limited to: proanthocyanidins, anthocyanins, procyanidins, catechins, flavones, flavone glycosides, lipoic acids, flavonoids, isoflavones, curcuminoids, stilbenoids, terpenes, carotenoids, rutosides, bithiazoles, pyrazolylthiazoles, benzoquinoliziums, xanthines, benzimidazoles, thiocyanates, isothiocyanates, omega-3 fatty acids and phenolic acids, with examples from this list such as astaxanthin or pycnogenol, alpha lipoic acid, resveratrol, pterostilbene, luteolin, quercetin, eicosapentaenoic acid, evodiamine and evodol and also included are molecules that correct and/or potentiate and/or increase (modulate) CFTR function such as certain plant polyphenols (e.g., flavones, flavonoids, isoflavones, curcuminoids, stilbenoids, terpenes, carotenoids, rutosides, bithiazoles, pyrazolylthiazoles, benzoquinoliziums, xanthines, benzimidazoles, thiocyanates, isothiocyanates and the like), and molecules such as forskolin that increase cyclic AMP, thereby activating CFTR, and molecules that are natural phosphodiesterase inhibitors that maintain CFTR function by maintaining cyclic AMP levels such as amentoflavone.

The goal of formulations derived from this invention is to maximize the potential to reduce inflammation in the body and to successfully modulate a wide range of CFTR mutations. Further laboratory research and feedback from patients will allow further refinement of formulations derived from this invention.

### REFERENCES

1. Alexeeff S E, Litonjua A A, Sparrow D, Vokonas P S, Schwartz J. Statin use reduces decline in lung function: VA Normative Aging Study. Am J Respir Crit Care Med. 2007 Oct. 15; 176(8):742-7. PubMed PMID: 17673694; PubMed Central PMCID: PMC2020828.
2. Li Y, Gottlieb J, Ma D, Kuehn C, Strueber M, Welte T, Simon A R, Warnecke G, Haverich A. Graft-protective effects of the HMG-CoA reductase inhibitor pravastatin after lung transplantation—a propensity score analysis with 23 years of follow-up. Transplantation. 2011 Aug. 27; 92(4):486-92. doi:10.1097/TP.0b013e318225670d. PubMed PMID: 21712756.
3. Dransfield M T, Wilhelm A M, Flanagan B, Courville C, Tidwell S L, Raju S V, Gaggar A, Steele C, Tang L P, Liu B, Rowe S M. Acquired cystic fibrosis transmembrane conductance regulator dysfunction in the lower airways in COPD. Chest. 2013 August; 144(2):498-506. doi: 10.1378/chest.13-O274. PubMed PMID: 23538783; PubMed Central PMCID: PMC3734887.
4. Lambert J A, Raju S V, Tang L P, McNicholas C M, Li Y, Courville C A, Farris R F, Coricor G E, Smoot L H, Mazur M M, Dransfield M T, Bolger G B, Rowe S M. Cystic fibrosis transmembrane conductance regulator activation by roflumilast contributes to therapeutic benefit in chronic bronchitis. Am J Respir Cell Mol Biol. 2014 March; 50(3):549-58. doi: 10.1165/rcmb.2013-O228OC. PubMed PMID: 24106801; PubMed Central PMCID: PMC4068936.
5. Ali E N, Mansour S Z. Boswellic acids extract attenuates pulmonary fibrosis induced by bleomycin and oxidative stress from gamma irradiation in rats. Chin Med. 2011 Sep. 30; 6:36. doi: 10.1186/1749-8546-6-36. PubMed PMID: 21961991; PubMedCentral PMCID: PMC3199276.
6. Hamdaoui N, Baudoin-Legros M, Kelly M, Aissat A, Moriceau S, Vieu D L, Colas J, Fritsch J, Edelman A, Planelles G. 2011. Resveratrol rescues cAMP-dependent anionic transport in the cystic fibrosis pancreatic cell line CFPAC1. Br J Pharmacol. June; 163(4):876-86.
7. Dhooghe B, Bouckaert C1, Capron A2, Wallemacq P2, Leal T1, Noel S3. 2015. Resveratrol increases F508del-CFTR dependent salivary secretion in cystic fibrosis mice. Biol Open. June 19; 4(7):929-36.
8. Lund, K. C.; Pantuso, T. (2014) Combination Effects of Quercetin, Resveratrol and Curcumin on In Vitro Intestinal Absorption. Journal of Restorative Medicine, Volume 3, Number 1, 1 April, pp. 112-120(9).
9. Ma T, Vetrivel L, Yang H, Pedemonte N, Zegarra-Moran O, Galietta L J, Verkman A S. High-affinity activators of cystic fibrosis transmembrane conductance regulator (CFTR) chloride conductance identified by high-throughput screening. 2002. J Biol Chem. October 4; 277(40):37235-41.
10. Caci E, Folli C, Zegarra-Moran O, Ma T, Springsteel M F, Sammelson R E, Nantz M H, Kurth M J, Verkman A S, Galietta L J. 2003. CFTR activation in human bronchial epithelial cells by novel benzoflavone and benzimidazolone compounds. Am J Physiol Lung Cell Mol Physiol. July; 285(1):L180-8.
11. Kandhare A D, Raygude K S, Ghosh P, Ghule A E, Bodhankar S L (2012). “Neuroprotective Effect of Naringin by Modulation of Endogenous Biomarkers in Streptozotocin Induced Painful Diabetic Neuropathy”. Fitoterapia. 83 (4): 650-9.
12. Kumar A, Dogra S, Prakash A (2010). “Protective Effect of Naringin, a Citrus Flavonoid, against Colchicine-Induced Cognitive Dysfunction and Oxidative Damage in Rats”. Journal of Medicinal Food. 13 (4): 976-84. 20673063.
13. Yang Z H1, Yu H J, Pan A, Du J Y, Ruan Y C, Ko W H, Chan H C, Zhou W L. (2008) Cellular mechanisms underlying the laxative effect of flavonol naringenin on rat constipation model. PLoS One. October 3; 3(10):e3348.
14. Pyle L C1, Fulton J C, Sloane P A, Backer K, Mazur M, Prasain J, Barnes S, Clancy J P, Rowe S M. (2010) Activation of the cystic fibrosis transmembrane conductance regulator by the flavonoid quercetin: potential use as a biomarker of ΔF508 cystic fibrosis transmembrane conductance regulator rescue. Am J Respir Cell Mol Biol. November; 43(5):607-16.
15. Zhang S1, Smith N, Schuster D, Azbell C, Sorscher E J, Rowe S M, Woodworth B A. (2011) Quercetin increases cystic fibrosis transmembrane conductance regulator-mediated chloride transport and ciliary beat frequency: therapeutic implications for chronic rhinosinusitis. Am J Rhinol Allergy. September-October; 25(5):307-12.
16. Flora K, Hahn M, Rosen H, et al. (1998) Milk thistle (Silybum marianum) for the therapy of liver disease. Am J Gastroenterol 93 (2): 139-43.
17. Blumenthal M, Busse W R, et al., eds. (1998) The Complete German Commission E Monographs: Therapeutic Guide to Herbal Medicines. Austin, Tex.: American Botanical Council.
18. Awatade N T1, Uliyakina I1, Farinha C M1, Clarke L A1, Mendes K1, Solé A2, Pastor J3, Ramos M M1, Amaral M D1. (2015) Measurements of Functional Responses in Human Primary Lung Cells as a Basis for Personalized Therapy for Cystic Fibrosis. EBioMedicine. 2014 Dec. 17; 2(2):147-53.

This invention is set forth in the following claims.

What is claimed is:

1. A composition for oral administration that is suitable for the treatment of an inflammatory condition in animals or humans, consisting of combinations of five or more natural and/or synthetic molecules that have anti-inflammatory and/or CFTR modulating functions wherein one or more compounds within the composition is capable of the following functions:
(1) Molecule(s) demonstrated to reduce inflammation in experimental models and/or in vivo.
(2) Molecule(s) demonstrated in experimental systems and/or in vivo to act as corrector(s) of CFTR.
(3) Molecule(s) demonstrated in experimental systems and/or in vivo to act as potentiator(s) of CFTR.
(4) Molecule(s) demonstrated in experimental systems and/or in vivo to increase levels of cyclic AMP.
(5) Molecule(s) demonstrated in experimental systems and/or in vivo to act as phosphodiesterase inhibitors.

2. A composition according to claim 1, wherein the compounds are selected from the classes of molecules consisting of: proanthocyanidins, anthocyanins, procyanidins, catechins, flavones, flavone glycosides, flavonoids, isoflavones, curcuminoids, lipoic acids, stilbenes and stilbenoids, terpenes and terpenoids, diterpenes, triterpenes, pentacyclic tri-terpenes, carotenoids, rutosides, bithiazoles, pyrazolylthiazoles, benzoquinoliziums, xanthines, benzimidazoles, thiocyanates, isothiocyanates, boswellic acids, saponins, omega-3 fatty acids and phenolic acids.

3. The composition of claim 1 where the disease in need of treatment is selected among: smoker's cough, inflammatory lung disease, pulmonary fibrosis, pulmonary vasculitis, pulmonary sarcoidosis, inflammation and/or infection associated with lung transplantation, acute lung rejection, burn wounds, chemical injury, military wound, pulmonary artery hypertension, bronchitis, sinusitis, asthma, pancreatitis, ocular inflammation, ocular infection, dry eye, cystic fibrosis, bacterial infection, fungal infection, parasite infection, viral infection, chronic obstructive pulmonary disease (COPD), vasculitis, sinus infection, irritable bowel syndrome (IBS), Crohn's disease, bronchiolitis obliterans syndrome (BOS), primary ciliary dyskinesia (PCD), alveolar proteinosis, idiopathic pulmonary fibrosis, familial pulmonary fibrosis, military wounds, burn wounds, eosinophilic pneumonia, eosinophilic bronchitis, acute lung injury, acute respiratory distress syndrome (ARDS), inflammation and/or infection associated with mechanical ventilation, ventilator-associated pneumonia, asbestos-related airway disorder or disease, dust-related airway disorder or disease, silicosis, chemical agent-related airway disease or disorder and any combination thereof.
